(19) United States
(12) Patent Application Publication
(10) Pub. No.: US 2009/0087488 A1
(43) Pub. Date: Apr. 2, 2009

(54) GALANTAMINE-CONTAINING CONTROLLED RELEASE ORAL DOSAGE FORMS, PROCESSES FOR THE PREPARATION THEREOF AND USE OF THE MANUFACTURE OF A MEDICAMENT

(76) Inventors: Rajan Kumar Verma, New Delhi (IN); Narayanan Badri Viswanathan, Chennai (IN); Rajeev Singh Raghuvanshi, Gurgaon (IN); Ashok Rampal, Amritsan (IN)

Correspondence Address: RANBAXY INC., 600 COLLEGE ROAD EAST, SUITE 2100, PRINCETON, NJ, 08540, US

(21) Appl. No.: 12/065,773

(22) PCT Filed: Sep. 1, 2006
(86) PCT No.: PCT/IB2006/002406

§ 371 Date: Oct. 23, 2008

(30) Foreign Application Priority Data
Sep. 5, 2005 (IN) 2376/DEL/2005

(51) Int. Cl. A61K 9/24; A61K 31/55

(52) U.S. Cl. 424/472; 514/215

(57) ABSTRACT

The present invention relates to controlled release oral dosage forms of galantamine or acceptable salts thereof and processes for the preparation thereof.

### TECHNICAL FIELD OF THE INVENTION

The present invention relates to controlled release oral dosage forms of galantamine or acceptable salts thereof and processes for the preparation thereof.

### BACKGROUND OF THE INVENTION

Galantamine is a known reversible, competitive acetylcholinesterase inhibitor. Chemically, it is 4aS,6R,8a-4-a,5,9,10,11,12-hexahydro-3-methoxy-11-methyl-6H-benzofuro[3a,3,2-ef[2]benzazepin-6-ol) and it is isolated from both the bulbs of the Caucasian snowdrops Galanthus woronowi and also from the common snowdrop Galanthus nivalis. Galantamine have been used as a curare reversal agent in anesthetic practice in Eastern bloc countries and also experimentally in the West, Galantamine and its salts have been tested in the treatment of mania, chronic fatigue syndrome, schizophrenia, alcoholism, nicotine dependence and Alzheimer's disease. Galantamine hydrobromide is currently available in tablets and capsules for use in the treatment of Alzheimer's disease as RAZADYNE™.

Galantamine has an affinity for nicotinic receptors but not for muscarinic receptors, and it is capable of passing the blood-brain barrier. Galantamine salts such as hydrobromide are given as immediate release tablets administered two or three times a day. The therapy with an immediate release dosage form leads to undesired peaks in the plasma profiles of galantamine and a sharp decrease in concentration after about 6 to 8 hours. These fluctuations ranging from high to low plasma concentrations are undesirable and may lead to side effects, such as nausea, vomiting or headaches. These side effects generally precipitate when galantamine is administered in high doses. A typical therapy with galantamine starts with a lower dose for 3-4 weeks and the dose is gradually increased till maximum tolerable dosage is achieved. If treatment with galantamine is interrupted for several days or longer, the patient will need to start over again at the lowest dose, increasing the dose at 4-week intervals until the former dose is achieved.

Currently galantamine is administered two or three times a day. It would be desirable to reduce the frequency of dosing to once a day which would result in reduced plasma fluctuations in the in the plasma concentration of galantamine. This objective may be achieved by administering galantamine in controlled release form such that the constant plasma levels of galantamine are maintained over an extended period of time.

WO 0038686 discloses a controlled release formulation containing galantamine as the active ingredient, wherein it includes particles comprising galantamine or a pharmaceutically acceptable acid addition salt thereof, a water soluble pharmaceutically acceptable excipient and optionally other pharmaceutically acceptable excipients. The particles are coated by a release rate controlling membrane coating. The water soluble excipient has been described as a water soluble polymer.

### SUMMARY OF THE INVENTION

In one general aspect there is provided a controlled release dosage form of galantamine. The controlled release dosage form includes a core comprising galantamine or a pharmaceutically acceptable salt and one or more of water insoluble excipients; and a rate-controlling coating.

Embodiments of the controlled release dosage form may include one or more of the following features. For example, the one or more water insoluble excipients may be one or more of silicon dioxide, ethylcellulose, crospovidone, sodium starch glycolate, crosscarmellose sodium, microcrystalline cellulose and methacrylate polymers.

The core may include an inert sphere onto which one or more water insoluble excipients are coated with galantamine dispersed in it. Alternatively, the core may include galantamine dispersed within it.

The rate-controlling coating may include one or more water insoluble polymers, a water soluble polymer and one or more plasticizers. The water insoluble polymer may be ethylcellulose, cellulose acetate, ammonio methacrylate copolymer and methacrylic acid copolymer. The water soluble polymer may be polyvinylpyrrolidone and hydroxypropylmethylcellulose. The one or more plasticizers may be triacetin, triethyl citrate, dibutyl phthalate, diethyl phthalate and polyethylene glycol.

The controlled release formulation may include a seal coat between the core and the release rate-controlling coating. The formulation may further include an immediate release portion which includes from about 10% to about 50% of the total dose of galantamine present. The immediate release portion may be a coating over the rate-controlling coating or as galantamine core devoid of rate-controlling coating.

In another general aspect there is provided a process for the preparation of controlled release formulation of galantamine. The process includes admixing galantamine or a pharmaceutically acceptable salt thereof with one or more water insoluble excipients to form a drug core; optionally applying a seal coat to the drug core; and applying a release-controlling coating over the core.

The one or more water insoluble excipients may be silicon dioxide, ethylcellulose, crospovidone, sodium starch glycolate, crosscarmellose sodium, microcrystalline cellulose and methacrylate polymers. The rate-controlling coating may include one or more water insoluble polymers, a water soluble polymer and one or more plasticizers. The water insoluble polymer may be ethylcellulose, cellulose acetate, ammonio methacrylate copolymer and methacrylic acid copolymer. The water soluble polymer may be polyvinylpyrrolidone and hydroxypropylmethylcellulose. The one or more plasticizers may be triacetin, triethyl citrate, dibutyl phthalate, diethyl phthalate and polyethylene glycol.

In another general aspect there is provided a method of treating alzheimer's dementia in a patient in need thereof. The method includes administering a controlled release dosage form of galantamine. The controlled release dosage form of galantamine includes a core comprising galantamine or a pharmaceutically acceptable salt and one or more of water insoluble excipients; and a rate-controlling coating.

Embodiments of the method may include one or more of the following features. For example, the one or more water insoluble excipients may be silicon dioxide, ethylcellulose, crospovidone, sodium starch glycolate, crosscarmellose sodium, microcrystalline cellulose and methacrylate polymers.

The rate-controlling coating may include one or more water insoluble polymers, a water soluble polymer and one or more plasticizers. The water insoluble polymer may be ethylcellulose, cellulose acetate, ammonio methacrylate copolymer and methacrylic acid copolymer. The water soluble polymer may be polyvinylpyrrolidone and hydroxypropylmethylcellulose. The one or more plasticizers may be triacetin, triethyl citrate, dibutyl phthalate, diethyl phthalate and polyethylene glycol

### DETAILED DESCRIPTION OF THE INVENTION

In the present invention the inventors have surprisingly found that a controlled release dosage form of galantamine may be prepared by using a core that includes galantamine and one or more water insoluble excipients, wherein the core is coated with a rate controlling layer.

These controlled release dosage forms provide steady plasma levels of galantamine over a period of 12 to 24 hours. The dosage forms may preferably be given once a day and would improve patient compliance.

Embodiments of the dosage form may include one or more of the following features. For example, the water insoluble excipients may include one or more of silicon dioxide, water insoluble polymers, such as ethylcellulose, crospovidone, sodium starch glycolate, cross-carmelose sodium, microcrystalline cellulose, water insoluble methacrylate polymers (ammonio methacrylate copolymer; e.g., Eudragit RLPO, Eudragit RSPO) and mixtures thereof. The water insoluble polymers may be present throughout the core or as a coating on a core that includes galantamine dispersed in it.

The core may be sugar spheres and be composed of sucrose, starch, mannitol, lactose, microcrystalline cellulose, sodium carboxymethyl cellulose, silica, and glass. Upon these cores a layer of galantamine may be dispersed along with one or more water insoluble polymer(s) and one or more pharmaceutically acceptable excipients to form a coating. Alternatively, the core may be prepared by conventional techniques known in the art including granulation, extrusion and spheronization and the galantamine may be dispersed within the core or coated onto the core.

The rate-controlling coating may include one or more water insoluble polymers. The water insoluble polymer may be ethylcellulose, cellulose acetate, ammonio methacrylate copolymers, and methacrylic acid copolymer. The rate controlling coating in addition to one or more water insoluble polymers may include one or more plasticizers, such as triethyl citrate, dibutyl phthalate, diethyl phthalate, triacetin, and PEG-400.

Optionally, in order to adjust the rate of release of galantamine the rate-controlling coating may include a water soluble polymer such as polyvinylpyrrolidone or hydroxypropyl methylcellulose. An optional seal coat may also be present between the core and the rate-controlling coating. The seal coat may include water soluble polymers such as hydroxypropyl methylcellulose.

These coated cores may be filled into capsules or compressed into tablets. Alternatively, the coated core may be formulated to include a unit dose of galantamine and administered as such.

In another embodiment, an immediate release portion sufficient to provide an initial therapeutic plasma levels may also be included in the dosage form. The immediate release portion may be cores of the invention devoid of rate-controlling coating or it may be a galantamine containing coating over the rate-controlling coating and is readily soluble in aqueous media. The immediate release portion may be at a concentration of between about 10% to about 50% of total dose of galantamine.

The term “galantamine” as defined herein includes galantamine base and pharmaceutically acceptable salts thereof. The pharmaceutically acceptable salt of galantamine may be the hydrobromide salt.

The controlled release dosage form may provide therapeutic plasma levels of galantamine from about 12 to about 24 hours. For example, the dosage form may be administered once a day.

Also provided in the present invention are processes for the preparation of controlled release dosage forms of galantamine. The process includes admixing galantamine or a pharmaceutically acceptable salt form thereof with one or more water insoluble excipients to form a drug core and applying the release rate controlling membrane coating.

Embodiments of the process include one or more of the following features. The process may include dispersing galantamine, the one or more water insoluble polymer(s), and one or more excipients in a pharmaceutically acceptable solvent system and coated on to the inert core by conventional coating processes. The coated core may then be further coated with a rate-controlling coating. An optional seal coat may also be coated prior to applying the rate-controlling coating.

In another embodiment of the process, galantamine may be mixed with one or more water insoluble excipients and granulated either with water alone or with a water insoluble binder solution. The granules are dried and may be compressed into cores and coated with the rate-controlling coating. In an alternative step, the process may include granulating a mixture of galantamine and one or more water insoluble excipients, extruding and spheronizing the wet mass into round spheroids, drying the spheroids and coating with the rate-controlling coating. An optional seal coat may also be coated prior to applying the rate-controlling coating.

In yet another embodiment of the process, galantamine may be sprayed onto the non-peril core and coated with a rate-controlling coating.

It is apparent from the text that various modifications and combinations of the formulations can be made without departing from the spirit and scope of the invention. For example, a water soluble or insoluble core may be used. Similarly, concentration of different excipients may vary as is apparent from the examples.

### EXAMPLE 1

| | | |
|---|---|---|
| S. No. | Ingredients | W/W % |

Core

| | | |
|---|---|---|
| 1. | Galantamine Hydrobromide | 8.91 |
| | | (eq. to 16 mg base) |
| 2. | Eudragit RLPO | 3.96 |
| 3. | Crospovidone | 3.96 |
| 4. | Colloidal silicon dioxide | 3.96 |
| 5. | Microcrystalline cellulose beads | 79.21 |
| 6. | Isopropyl alcohol water mixture (75:25) | q.s. |

Rate controlling coating

| | | |
|---|---|---|
| 6. | Ethyl cellulose 20 cps | 65.4 |
| 7. | Hydroxypropylmethylcellulose | 28.0 |
| 8. | Triacetin | 6.6 |
| 9. | Isopropyl alcohol water mixture (90:10) | q.s. |

### Procedure:

1. Galantamine hydrobromide, Eudragit RLPO, crospovidone and colloidal silicon dioxide were dispersed in isopropyl alcohol/water mixture (75/25).
2. Microcrystalline cellulose beads were loaded in a fluid bed coating apparatus and sprayed with dispersion of step 1.
3. The above drug layered cores were coated with the coating composition which includes ethyl cellulose, hydroxypropylmethylcellulose and triacetin dissolved in an isopropyl alcohol/water mixture (90/10 weight by weight).

The cores with varying percentages of rate controlling coating were prepared i.e. beads had rate controlling coating in amounts of 8.8%; 10%; 11%, and 12% w/w.

The coated cores of Example 1 were subjected to dissolution studies with USP II dissolution apparatus in 900 mL phosphate buffer of pH 6.8 at 50 rpm. The results are given in Table 1.

TABLE 1
Dissolution profile of coated cores of Example 1 with varying
amounts of rate controlling coating in USP II dissolution
apparatus in 900 mL phosphate buffer of pH 6.8 at 50 rpm.

% Drug released for specific
amount of release controlling coating

| Time (Hrs.) | 8.8% | 10% | 11% | 12% |
|---|---|---|---|---|
| 0 | 0 | 0 | 0 | 0 |
| 1 | 18 | 11 | 10 | 8 |
| 2 | 46 | 31 | 31 | 25 |
| 4 | 78 | 63 | 65 | 58 |
| 6 | 88 | 80 | 83 | 79 |
| 8 | 92 | 88 | 90 | 87 |
| 12 | 94 | 93 | 96 | 95 |
| 16 | 94 | 95 | 96 | 97 |
| 20 | 95 | 95 | 97 | 99 |
| 24 | 94 | 96 | 96 | 98 |

### EXAMPLE 2

| | | |
|---|---|---|
| S. No. | Ingredients | W/W % |

Core

| | | |
|---|---|---|
| 1. | Galantamine Hydrobromide | 8.66 |
| | | (eq. to 16 mg base) |
| 2. | Eudragit RLPO | 7.46 |
| 3. | Microcrystalline cellulose beads | 83.89 |
| 4. | Isopropyl alcohol water mixture (50:50) | q.s. |

Rate controlling coating

| | | |
|---|---|---|
| 4. | Ethyl cellulose 20 cps | 59.6 |
| 5. | Hydroxypropylmethylcellulose | 25.6 |
| 6. | Diethyl phthalate | 14.8 |
| 7. | Isopropyl alcohol water mixture (90:10) | q.s. |

### Procedure:

1. Galantamine hydrobromide and Eudragit RLPO were dispersed in an isopropyl alcohol/water mixture (50/50 weight by weight).
2. Microcrystalline cellulose beads were loaded in a fluid bed coating apparatus and sprayed with dispersion of step 1.
3. The above drug layered cores were coated with the coating composition containing ethyl cellulose, hydroxypropylmethylcellulose and diethyl phthalate dissolved in isopropyl alcohol/water mixture (90/10 weight by weight).

The cores with varying percentages of rate controlling coating were prepared i.e. beads had rate controlling coating in amounts of 6.4%, 9%, and 11.3% w/w.

The coated cores of Example 2 were subjected to dissolution studies with USP II dissolution apparatus in 900 mL phosphate buffer of pH 6.8 at 50 rpm. The results are given in Table 2.

TABLE 2
Dissolution profile of coated cores of Example 2 with varying
amounts of rate controlling coating in USP II dissolution
apparatus in 900 mL phosphate buffer of pH 6.8 at 50 rpm.

| | % Drug released for specific | | |
|---|---|---|---|
| | amount of release controlling | | |
| Time | coating | | |
| (Hrs.) | 6.4% | 9% | 11.3% |
| 0 | 0 | 0 | 0 |
| 1 | 33 | 14 | 8 |
| 2 | 70 | 42 | 29 |
| 4 | 99 | 84 | 67 |
| 6 | 106 | 100 | 89 |
| 8 | 108 | 107 | 99 |
| 12 | 109 | 111 | 107 |
| 16 | 109 | 111 | 108 |
| 24 | 108 | 111 | 109 |

### EXAMPLE 3

| | | |
|---|---|---|
| S. No. | Ingredients | W/W % |

Core

| | | |
|---|---|---|
| 1. | Galantamine Hydrobromide | 8.66 |
| | | (eq. to 16 mg base) |
| 2. | Eudragit RLPO | 7.46 |
| 3. | Microcrystalline cellulose beads | 83.89 |
| 4. | Isopropyl alcohol water mixture (50:50) | q.s. |

Rate controlling coating

| | | |
|---|---|---|
| 4. | Ethyl cellulose 7 cps | 65.4 |
| 5. | Hydroxypropylmethylcellulose | 24.8 |
| 6. | Diethyl phthalate | 9.8 |

### Procedure:

1. Galantamine hydrobromide and Eudragit RLPO were dispersed in isopropyl alcohol/water mixture (50/50 weight by weight).
2. Microcrystalline cellulose beads were loaded in a fluid bed coating apparatus and sprayed with dispersion of step 1.
3. The above drug layered cores were coated with the coating composition that includes ethyl cellulose, hydroxypropylmethylcellulose and diethyl phthalate dissolved in an isopropyl alcohol/water mixture (90/10 weight by weight).

The cores with varying percentages of rate controlling coating were prepared i.e. beads had rate controlling coating in amounts of 9%, 10%, 11%, and 12% w/w.

The coated cores of Example 3 were subjected to dissolution studies with USP II dissolution apparatus in 900 mL phosphate buffer of pH 6.8 at 50 rpm. The results are given in Table 3.

TABLE 3
Dissolution profile of coated cores of example 3 with varying
amounts of rate controlling coating in USP II dissolution
apparatus in 900 mL phosphate buffer of pH 6.8 at 50 rpm.

% Drug released for specific
amount of release controlling coating

| Time (Hrs) | 9% | 10% | 11% | 12% |
|---|---|---|---|---|
| 0 | 0 | 0 | 0 | 0 |
| 1 | 6 | 3 | 3 | 2 |
| 2 | 22 | 15 | 12 | 8 |
| 4 | 54 | 47 | 39 | 28 |
| 6 | 75 | 69 | 63 | 49 |
| 8 | 87 | 83 | 78 | 67 |
| 12 | 96 | 96 | 94 | 88 |
| 16 | 99 | 100 | 100 | 97 |
| 24 | 100 | 101 | 102 | 103 |

### EXAMPLE 4

| | | |
|---|---|---|
| S. No. | Ingredients | W/W % |

Core

| | | |
|---|---|---|
| 1. | Galantamine Hydrobromide | 8.66 |
| | | (eq. to 16 mg base) |
| 2. | Eudragit RLPO | 7.46 |
| 3. | Microcrystalline cellulose beads | 83.89 |
| 4. | Isopropyl alcohol water mixture (50:50) | q.s. |

Rate controlling coating

| | | |
|---|---|---|
| 4. | Ethyl cellulose 10 cps | 65.4 |
| 5. | Hydroxypropylmethylcellulose | 24.8 |
| 6. | Diethyl phthalate | 9.8 |
| 7. | Isopropyl alcohol water mixture (90:10) | q.s. |

### Procedure:

1. Galantamine hydrobromide and Eudragit RLPO were dispersed in an isopropyl alcohol/water mixture (50/50 weight by weight).
2. Microcrystalline cellulose beads were loaded in a fluid bed coating apparatus and sprayed with dispersion of step 1.
3. The above drug layered cores were coated with the coating composition that includes ethyl cellulose, hydroxypropylmethylcellulose and diethyl phthalate dissolved in an isopropyl alcohol/water mixture (90/10 weight by weight).

The cores with varying percentages of rate controlling coating were prepared i.e. beads had rate controlling coating in amounts of 5.1%, 6.1%, and 6.4% w/w.

The coated cores of Example 4 were subjected to dissolution studies with USP II dissolution apparatus in 900 mL phosphate buffer of pH 6.8 at 50 rpm. The results are given in Table 4.

TABLE 4
Dissolution profile of coated cores of example 4 with varying
amounts of rate controlling coating in USP II dissolution
apparatus in 900 mL phosphate buffer of pH 6.8 at 50 rpm.

| | % Drug released for | | |
|---|---|---|---|
| | specific amount of | | |
| | release controlling coating | | |
| Time (Hrs.) | 5.1% | 6.1% | 6.4% |
| 0 | 0 | 0 | 0 |
| 1 | 7 | 4 | 3 |
| 2 | 22 | 15 | 10 |
| 4 | 53 | 40 | 32 |
| 6 | 75 | 61 | 52 |
| 8 | 86 | 75 | 68 |
| 12 | 97 | 90 | 86 |
| 16 | 100 | 94 | 94 |
| 24 | 102 | 97 | 98 |

### EXAMPLE 5

| | | |
|---|---|---|
| S. No. | Ingredients | W/W % |

Core

| | | |
|---|---|---|
| 1. | Galantamine Hydrobromide | 8.91 |
| | | (eq. to 16 |
| | | mg base) |
| 2. | Eudragit RLPO | 3.96 |
| 3. | Crospovidone | 3.96 |
| 4. | Colloidal silicon dioxide | 3.96 |
| 5. | Non-peril sugar beads (18-20 mesh fractions) | 79.21 |
| 6. | Isopropyl alcohol water mixture (75:25) | q.s. |

Rate controlling coating

| | | |
|---|---|---|
| 6. | Ethyl cellulose 20 cps | 69.76 |
| 7. | Hydroxypropylmethylcellulose | 23.24 |
| 8. | Triacetin | 6.96 |
| 9. | Isopropyl alcohol water mixture (90:10) | q.s. |

### Procedure:

1. Galantamine hydrobromide, Eudragit RLPO, crospovidone and colloidal silicon dioxide were dispersed in an isopropyl alcohol/water mixture (75/25 weight by weight).
2. Non-peril sugar beads were loaded in a fluid bed coating apparatus and sprayed with dispersion of step 1.
3. The above drug layered cores were coated with the coating composition that includes ethyl cellulose, hydroxypropylmethylcellulose and triacetin dissolved in an isopropyl alcohol/water mixture (90/10 weight by weight). The beads were coated to a weight gain of 5.3% w/w.

The coated cores of Example 5 were subjected to dissolution studies with USP II dissolution apparatus in 900 mL phosphate buffer of pH 6.8 at 50 rpm. The results are given in Table 5.

TABLE 5
Dissolution profile of coated cores of Example 5 in USP II dissolution
apparatus in 900 mL phosphate buffer of pH 6.8 at 50 rpm.

| | % Drug released for |
|---|---|
| | 5.3% w/w of release |
| Time (Hrs.) | controlling coating |
| 0 | 0 |
| 1 | 4 |
| 2 | 20 |
| 4 | 40 |
| 8 | 65 |
| 12 | 74 |
| 16 | 79 |
| 24 | 84 |

### EXAMPLE 6

### Preparation of Spheroids

| S. No. | Ingredients | % w/w |
|---|---|---|
| 1 | Galantamine Hydrobromide | 11.4 |
| | | (eq. to 16 mg base) |
| 2 | Crospovidone | 9.9 |
| 3 | Silicon dioxide | 4.9 |
| 4 | Microcrystalline cellulose | 73.8 |
| 5. | Water | q.s. |

1. All the ingredients were blended in a granulator.
2. Water was added to the above blend and mixed till a wet mass was obtained.
3. The wet mass was extruded to give cylindrical extrudes.
4. The damp extrudes were placed in spheronizer and rotated till spheroids were formed.
5. The resultant spheroids were dried in fluid bed drier at 60° C.
6. The dried spheroids were screened to give fraction between #16-25 BSS screen.

The above spheroids were coated with following coating composition:

| S. No. | Ingredients | % w/w |
|---|---|---|
| 1 | Ethyl cellulose - 20 cps | 59.58 |
| 2 | hydroxypropylmethylcellulose | 25.52 |
| 3 | Triacetin | 14.9 |
| 4. | Isopropyl alcohol water mixture (90:10) | q.s. |

### Procedure

1. All the ingredients were dissolved in an isopropyl alcohol-water mixture (90/10 weight by weight).
2. Spheroids were placed in fluid-bed coating apparatus and coated with above coating solution.

The spheroids with varying percentages of rate controlling coating were prepared i.e. spheroids had rate controlling coating in amounts of 6.7%, 7.5%, 8.0%, and 8.6% w/w.

The coated spheroids of Example 6 were subjected to dissolution studies with USP II dissolution apparatus in 900 mL phosphate buffer of pH 6.8 at 50 rpm. The results are given in Table 6.

TABLE 6
Dissolution profile of coated spheroids of Example 6 with varying
amounts of rate controlling coating in USP II dissolution apparatus
in 900 mL phosphate buffer of pH 6.8 at 50 rpm.

% Drug released for specific amount
of release controlling coating

| Time (Hrs.) | 6.7% | 7.5% | 8% | 8.6% |
|---|---|---|---|---|
| 0 | 0 | 0 | 0 | 0 |
| 1 | 24 | 18 | 16 | 13 |
| 2 | 47 | 37 | 34 | 28 |
| 4 | 71 | 61 | 58 | 50 |
| 6 | 83 | 74 | 71 | 65 |
| 8 | 89 | 81 | 80 | 75 |
| 12 | 94 | 89 | 89 | 87 |
| 16 | 96 | 92 | 94 | 92 |
| 24 | 97 | 95 | 97 | 97 |

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

We claim:

1. A controlled release dosage form of galantamine comprising:
a. a core comprising galantamine or a pharmaceutically acceptable salt and one or more of water insoluble excipients; and
b. a rate-controlling coating.

2. The controlled release formulation according of claim 1, wherein the one or more water insoluble excipients comprise silicon dioxide, ethylcellulose, crospovidone, sodium starch glycolate, crosscarmellose sodium, microcrystalline cellulose and methacrylate polymers.

3. The controlled release formulation according to claim 1, wherein the core comprises an inert sphere onto which one or more water insoluble excipients is coated with galantamine dispersed in it.

4. The controlled release formulation according to claim 1, wherein the core comprises galantamine dispersed within it.

5. The controlled release formulation according to claim 1, wherein the rate-controlling coating comprises one or more water insoluble polymers, a water soluble polymer and one or more plasticizers.

6. The controlled release formulation according to claim 5, wherein the water insoluble polymer comprises ethylcellulose, cellulose acetate, ammonio methacrylate copolymer and methacrylic acid copolymer.

7. The controlled release formulation according to claim 5, wherein the water soluble polymer comprises polyvinylpyrrolidone and hydroxypropylmethylcellulose.

8. The controlled release formulation according to claim 5, wherein the one or more plasticizers comprise triacetin, triethyl citrate, dibutyl phthalate, diethyl phthalate and polyethylene glycol.

9. The controlled release formulation according to claim 1, wherein a seal coat lies between the core and the release rate-controlling coating.

10. The controlled release formulation according to claim 1, further comprising an immediate release portion comprising from about 10% to about 50% of the total dose of galantamine present.

11. The controlled release formulation according to claim 10, wherein the immediate release portion comprises a coating over the rate-controlling coating or as galantamine core devoid of rate-controlling coating.

12. A process for the preparation of controlled release formulation of galantamine, the process comprising:
a. admixing galantamine or a pharmaceutically acceptable salt thereof with one or more water insoluble excipients to form a drug core;
b. optionally applying a seal coat to the drug core; and
c. applying a release-controlling coating over the core.

13. The process according of claim 12, wherein the one or more water insoluble excipients comprise silicon dioxide, ethylcellulose, crospovidone, sodium starch glycolate, crosscarmellose sodium, microcrystalline cellulose and methacrylate polymers.

14. The process according to claim 12, wherein the rate-controlling coating comprises one or more water insoluble polymers, a water soluble polymer and one or more plasticizers.

15. The process according to claim 14, wherein the water insoluble polymer comprises ethylcellulose, cellulose acetate, ammonio methacrylate copolymer and methacrylic acid copolymer.

16. The process according to claim 14, wherein the water soluble polymer comprises polyvinylpyrrolidone and hydroxypropylmethylcellulose.

17. The process according to claim 14, wherein the one or more plasticizers comprise triacetin, triethyl citrate, dibutyl phthalate, diethyl phthalate and polyethylene glycol.

18. A method of treating alzheimer's dementia in a patient in need thereof, the method comprising administering a controlled release dosage form of galantamine comprising:
a. a core comprising galantamine or a pharmaceutically acceptable salt and one or more of water insoluble excipients; and
b. a rate-controlling coating.

19. The method according of claim 18, wherein the one or more water insoluble excipients comprise silicon dioxide, ethylcellulose, crospovidone, sodium starch glycolate, crosscarmellose sodium, microcrystalline cellulose and methacrylate polymers.

20. The method according to claim 18, wherein the rate-controlling coating comprises one or more water insoluble polymers, a water soluble polymer and one or more plasticizers.

21. The method according to claim 18, wherein the water insoluble polymer comprises ethylcellulose, cellulose acetate, ammonio methacrylate copolymer and methacrylic acid copolymer.

22. The method according to claim 18, wherein the water soluble polymer comprises polyvinylpyrrolidone and hydroxypropylmethylcellulose.

23. The method according to claim 1S, wherein the one or more plasticizers comprise triacetin, triethyl citrate, dibutyl phthalate, diethyl phthalate and polyethylene glycol.
